(19) United States
(12) Patent Application Publication
(10) Pub. No.: US 2012/0221053 A1
(43) Pub. Date: Aug. 30, 2012

(54) Spine Fixation System

(75) Inventor: Franz Copf, Stuttgart (DE)

(73) Assignee: SPONTECH SPINE INTELLIGENCE GROUP AG, Stuttgart (DE)

(21) Appl. No.: 13/387,949

(22) PCT Filed: Jul. 19, 2010
(86) PCT No.: PCT/EP2010/004377

§ 371 Date: May 14, 2012

(30) Foreign Application Priority Data
Jul. 30, 2009 (EP) 09009847.6

(51) Int. Cl. A61B 17/70

(52) U.S. Cl. 606/251

(57) ABSTRACT

A spine fixation system (10; 210; 310) comprises a first rod (16; 216) that connects a first vertebra (V2) to a second vertebra (V) but not to a third vertebra (V4), and a second rod (14; 214) that connects the second vertebra (V3) to the third vertebra (V4) but not to the first vertebra. The spine fixation system is configured such that first vertebra (V2), but not the third vertebra (V4), is allowed to move relative to the second (V3) vertebra after the spine fixation system has been completely implanted.

### BACKGROUND OF THE INVENTION

1. Field of the Invention

The present invention relates to a spine fixation system for the surgical treatment of spinal disorders which may require correction, stabilization, adjustment or fixation of the spinal column.

2. Description of Related Art

Various types of spinal column disorders are known and include scoliosis (abnormal curvature or rotation of vertebrae relative to the plane of the spine), kyphosis (abnormal backward curvature of the spine) and spondylolisthesis (forward displacement of a lumber vertebra), all of which involve a “misalignment” of the spinal column. Patients who suffer from such conditions usually experience extreme, debilitating pain and physical deformity due to the condition. In severe cases treatments for these conditions have used a technique known as fusion with spinal fixation which results in the mechanical immobilization of areas of the spine and the eventual fusion of the vertebrae in the regions treated. In less severe cases treatment comprises decompression of the affected nerves and fusion of the vertebrae involved.

Fusion, however, is not usually successful unless the vertebrae are also fixed for a time period by a mechanical device installed internally during surgery. This allows the fused bone time to heal. Numerous mechanical systems have been proposed for this purpose. Screw and rod systems and screw and plate systems are commonly used to this purpose. The former system typically uses a rigid rod secured to the spine by screws inserted in the pedicles for holding the rod. The rod may be bent to the desired configuration, and this both manipulates and holds the vertebrae in that same configuration until the fusion process can permanently accomplish the same thing.

During the last few years more and more non-fusion implants have been implanted in cases where fusion implants had been used formerly. A non-fusion implant maintains, at least to a certain extent, the mobility of the adjacent vertebrae. Non-fusion implants usually comprise two plates, which are in contact to the adjacent vertebrae, and a joint, for example a ball-and-socket joint, which is arranged between the plates and enables their relative movements. However, even non-fusion implants often require some kind of stabilization until the two plates are rigidly connected to the adjacent vertebrae as a result of bone growth. For such spine fixation systems the use of flexible rods has been proposed. The flexible rods have such a low bending stiffness that the adjacent vertebrae are allowed to perform, at least to a certain extent, relative movements.

In some cases the spinal column of a patient requires some vertebrae to be fused and some to be connected by a non-fusion implant. At present two (or even more) surgeries are usually performed in such cases. In one surgery the fusion implants are inserted and a rigid spine fixation system is implanted. In the other surgery the non-fusion implants are inserted and a flexible spine fixation system is implanted. This approach is unsatisfactory because the patient has to be subjected to two surgeries, and the time required for recovering from the two surgeries is very significant.

### SUMMARY OF THE INVENTION

It is therefore an object to provide a spine fixation system which makes it possible to fix vertebrae such that some vertebrae are allowed to perform, at least to a certain extent, relative movements and others are not.

According to the invention, this object is achieved by an implanted spine fixation system comprising a first rod that connects a first vertebra to a second vertebra but not to a third vertebra, and a second rod that connects the second vertebra to the third vertebra but not to the first vertebra. The spine fixation system is configured such that first vertebra, but not the third vertebra, is allowed to move relative to the second vertebra after the spine fixation system has been completely implanted.

Then the first rod forms a flexible connection to an adjacent vertebra which is separated by a non-fusion implant, and the second rod forms a non-flexible connection to an adjacent vertebra which is separated by a fusion implant. The second rod forming a connection to the fused vertebra will usually be rigid, whereas the first rod connecting to the non-fused vertebra is flexible as such, which means that the first rod has a smaller bending stiffness than the second rod, and/or is flexibly connected to the vertebra.

In the former case the positions of the two seat members may be, after the spine fixation system has been completely implanted in the human body, fixed with regard to the fastener to which the connector is attached. In this case the different flexibility is exclusively caused by the different bending stiffness of the two rods. The bending stiffness is defined as the product of the area moment of inertia of the rods cross-section and its elastic modulus. Thus two rods having a different bending stiffness may be made of the same material, but may have differing cross-sections, or may have equal cross-sections but are made of different materials, or may differ with regard to the cross-section and also the elastic modulus.

In the latter case the system may comprise fasteners, which are secured to the vertebrae, and the first rod is flexibly connected to fasteners that are secured to the first and second vertebrae.

This may be achieved by providing connectors, which are attached to the fasteners and comprise seat members that are connected to the rods. The position of the seat members that are connected to the first rod are, after the spine fixation system has been completely implanted in a human body (i.e. after completion of the implant surgery in the human body), allowed to change in response to forces exerted by the first and second vertebrae.

The fasteners may be configured to be secured to a pedicle of the vertebra to be treated. In some embodiments the fasteners are configured to be cemented into a bore in the respective vertebra. In other embodiments the fasteners are screws.

In its non-implanted state, a spine fixation system in accordance with the present invention comprises:
a) two rods which are configured to extend over a portion of the spine,
b) a plurality of fasteners wherein each fastener has a longitudinal axis and is configured to be secured to a vertebra to be treated,
c) a connector which
   is attached to, or is capable of being attached to, one of the fasteners and
   comprises two seat members each being configured to be connected to one of the two rods, wherein
      the position of at least one of the two seat members relative to the fastener, to which the connector is attached, is, after the spine fixation system has been completely implanted (i.e. after completion of the implant surgery in the human body), allowed to change in response to forces exerted by the portion of the spine.

Such a flexible position of the at least one seat member enables the rod connecting vertebrae which are separated by a non-fusion implant to perform relative movements with regard to these vertebrae. The term “change of position” encompasses any arbitrary movement, in particular rotations around arbitrary axes and translational displacements along arbitrary directions and combinations of such movements.

To this end the connector may comprise a joint that allows changes of the position of the at least one seat member after the spine fixation system has been completely implanted in the human body. The joint may be provided between the seat member and another component of the connector and/or may be provided between two components of the connector from which one supports the at least one seat member.

The at least one seat member may be allowed to perform rotational movements about at least two different axes with regard to the fastener to which the connector is attached. This takes into account that the main relative movements between two vertebrae (inclination/reclination and lateral flexion) may be described as rotations around two substantially orthogonal axes. Therefore it is preferred if the at least two different axes are orthogonal to each other. As a matter of course, rotations about a third (preferably orthogonal) rotational axis may also be permitted.

In one embodiment the at least one seat member is allowed to perform rotational movements about a first axis and a second axis. The connector comprises a body portion, through which the longitudinal axis of the fastener, to which the connector is connected, extends. The connector further comprises an arm member which projects from the body portion and supports the at least one seat member. The first axis extends through the at least one seat member and the arm member and the second axis extends through the arm member and the body portion. This will usually imply that different joints are provided each of which enabling rotational movements around one rotational axis. Having two different joints usually permits a different range of movements than would be obtained with a single joint, for example a ball bearing.

In one embodiment the spine fixation system comprises a (preferably adjustable) range delimiter which is configured to delimit a range of allowed position changes. This is useful for preventing undue strains on the surrounding ligaments, muscles and other tissue. An adjustability may be achieved by a range delimiter which comprises an element which has an impact on the range of allowed position changes. If a set of different elements is provided, from which the surgeon can select a suitable one when he assembles the spine fixation system, he is able to determine the range of allowed positions. Such an element may be, for example, an insert having a groove with a certain length which determines the range of allowed position changes.

In another embodiment the spine fixation system comprises a (preferably adjustable) restoring force member which is configured to exert a restoring force acting against the forces exerted by the portion of the spine and causing a position change of the at least one seat member. The healthy intervertebral disc also produces restoring forces if the adjacent vertebrae are moved. A spine fixation system having a restoring force member enables such restoring forces. An adjustability may be achieved by a replaceable restoring force member. To this end a set of different restoring force members may be provided having different restoring force characteristics, i.e. having different dependencies of the restoring force on the position change. In the simplest case the restoring force member is an element made of a resilient material which may be subjected to torsion, compression or strain, for example.

At least one of the two seat members may be capable of being fixed in different rotational positions with regard to a rotational axis that is, for enabling the at least one seat member to be polyaxially adjusted, capable of being fixed in different tilting positions at least within a cone of tilting angles.

The rod is then allowed to perform, together with the seat member, variable tilting movements whilst remaining fully received (i.e. with sufficient contact surfaces) in the seat member. Without a polyaxial adjustability it will often not be possible to insert the rod in the seat member without moving the vertebrae to undesired positions.

The connector may be fixedly attached to the fastener, or may even be integrally formed therewith. In a preferred embodiment, however, each connector is configured such that it can be connected to the respective fastener after the fastener has been secured to the vertebra to be treated. This facilitates the implant of the fastener into the vertebra because no connector obstructs the way for inserting a suitable tool. Once the fastener is implanted, the connector is attached to the fastener and finally fixed.

The seat member may have a recess for receiving the rod. This recess may, in its cross-section, be U-shaped which results in a tulip-like seat member. At the open end of the U-shaped recess the rod can be easily inserted and then be fixed with the help of a clamp mechanism, for example a fixing screw. The recess may still enable axial displacement of the rod within the recess for performing final adjustments of the rod within a row of seat members arranged one behind the other along a human spinal column.

In a preferred embodiment the position of one of the two seat members is, after the spine fixation system has been completely implanted in the human body, fixed with regard to the fastener to which the connector is attached. The position of the other of the two seat members is, after the spine fixation system has been completely implanted in the human body, allowed to change in response to forces exerted by the portion of the spine. In such a configuration the connector is ideally suited to be fastened to a vertebra which is, on one side, separated to an adjacent vertebra by a fusion implant, and to the other adjacent vertebra by a non-fusion implant.

In another preferred embodiment the position of both seat members is, after the spine fixation system has been completely implanted in the human body, allowed to change in response to forces exerted by the portion of the spine. In such a configuration the connector is ideally suited to be fastened to a vertebra which is, on one side, separated to both adjacent vertebrae by a non-fusion implant.

According to yet another embodiment the spine system comprises a further connector which is attached to or is capable of being attached to, one of the fasteners and which comprises exactly one seat member being configured to be connected to one of the two rods. The position of the seat member is, after the spine fixation system has been completely implanted in the human body, fixed with regard to the fastener to which the further connector is attached. Such a further connector having only one seat member may be used for those vertebrae which are connected, on each side of the vertebrae, to only one rod.

A still further connector may be provided which is attached to, or is capable of being attached to, one of the fasteners and which also comprises exactly one seat member being configured to be connected to one of the two rods. However, with this still further connector the position of the seat member is, after the spine fixation system has been completely implanted in the human body, allowed to change in response to forces exerted by the portion of the spine. Such a connector may be used for vertebrae which are, on each side, connected only to one rod, but are separated to an adjacent vertebra by a non-fusion implant so that a flexible seat member is required.

### BRIEF DESCRIPTION OF THE DRAWINGS

Various features and advantages of the present invention may be more readily understood with reference to the following detailed description taken in conjunction with the accompanying drawing in which:

FIG. 1 is a perspective view of a spine fixation system according to a first embodiment of the invention;

FIG. 2 is a sectional view through an upper portion of a connector which is part of the spine fixation system shown in FIG. 1;

FIG. 3 is a schematic top view of a segment of a human spine in which the spine fixation system shown in FIGS. 1 and 2 has been implanted;

FIG. 4 is a schematic top view similar to FIG. 3, but with two adjacent vertebrae in a state of lateral flexion;

FIG. 5 is a side view of a portion of the spine segment shown in FIGS. 3 and 4 in a neutral position of two adjacent vertebrae;

FIG. 6 is a side view similar to FIG. 5, but with the two vertebrae being in an inclined position;

FIG. 7 is a schematic top view of a segment of a human spine in which two non-fusion implants and a spine fixation system according to the first embodiment have been implanted;

FIG. 8 is a perspective view of a spine fixation system according to a second embodiment of the invention;

FIG. 9 is sectional view through an upper portion of a connector which is part of the spine fixation system shown in FIG. 8;

FIG. 10 is a top/bottom view of a resilient member which is part of a resilient joint formed between a cylindrical portion and a projection of the connector shown in FIG. 9;

FIG. 11 is a bottom view of a resilient ring arranged in a second seat member of the connector shown in FIG. 9;

FIG. 12 is a schematic top view of a segment of a human spine in which the spine fixation system shown in FIGS. 8 to 11 has been implanted, wherein two adjacent vertebrae are in a state of lateral flexion;

FIG. 13 is a side view of a portion of the spine segment shown in FIG. 12 in a neutral position of two adjacent vertebrae;

FIG. 14 is a side view similar to FIG. 13, but with the two vertebrae being in an inclined position;

FIG. 15 is a schematic top view of a segment of a human spine in which two non-fusion implants and a spine fixation system according to the third embodiment have been implanted;

FIG. 16 is sectional view through an upper portion of a connector which is part of the spine fixation system shown in FIG. 15;

FIG. 17 is a side view on a projection of the connector shown in FIG. 16;

FIG. 18 is a side view on an insert which may be inserted into the projection shown in FIG. 17.

### DESCRIPTION OF PREFERRED EMBODIMENTS

### 1. First Embodiment

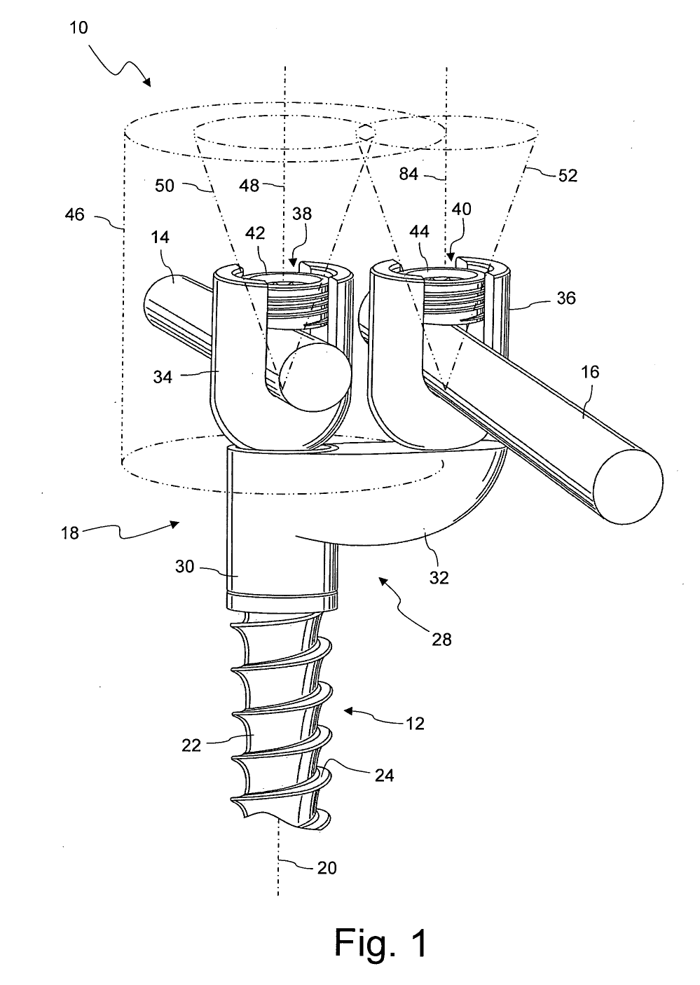

FIG. 1 is a perspective view of important components of a spine fixation system 10 according to a first embodiment in an assembled state. These main components are a pedicle screw 12, a first rod 14, a second rod 16 and a connector 18 which is configured to connect the rods 14, 16 to the pedicle screw 12.

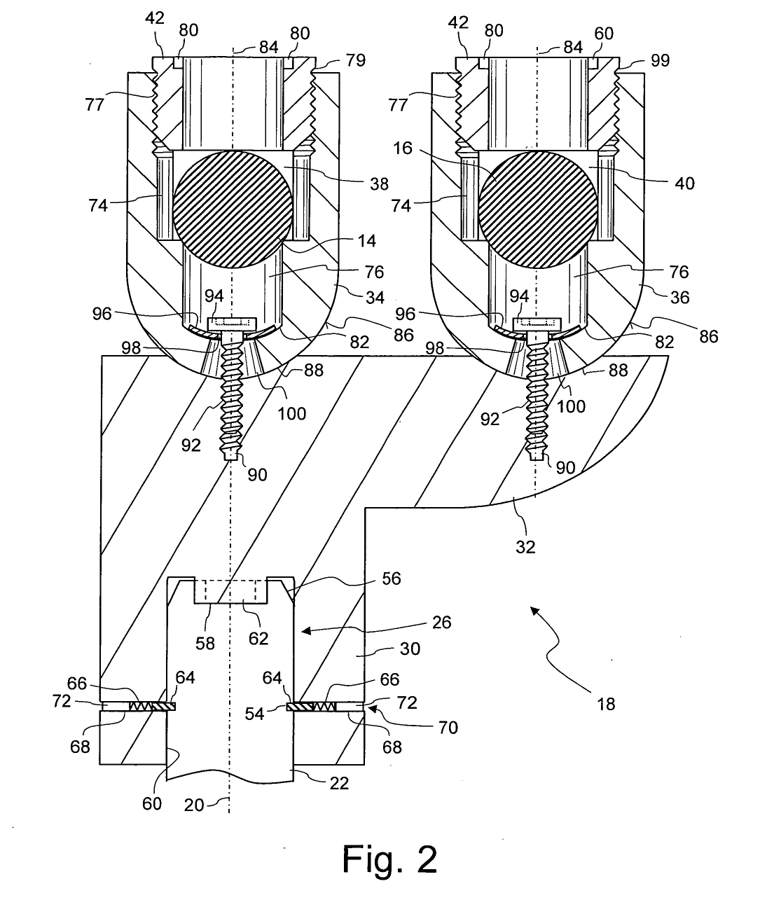

The pedicle screw 12 has a longitudinal axis 20 and an at least substantially cylindrical portion 22 supporting an external thread 24. At its free end the pedicle screw 12 may have a conical tip (not shown) and at its opposite end a screw head 26 which can, because it is covered by the connector 18, only be seen in the sectional view of FIG. 2 that will be explained further below.

The pedicle screw 12 is configured with regard to its length, diameter and the external thread 24 such that it can be screwed into the pedicle of a human spine. However, not only screws but other types of fasteners may be used to this end. Such fasteners include, but are not limited to, bolts having ridges on their outer surfaces so that the bolts can be cemented into cylindrical bores drilled in the pedicles of the vertebrae to be treated.

The connector 18 comprises a head member 28 including a cylindrical portion 30 and a projection 32 which extends radially away from the longitudinal axis 20 and is, in the embodiment shown, integrally formed with the cylindrical portion 30. The head member 28 supports a first seat member 34, to which the first rod 14 can be secured, and a second seat member 36, to which the second rod 16 can be secured. Each of the seat members 34, 36 is formed as a tulip comprising a recess 38 and 40, respectively, that are configured to receive the rods 14, 16. At an upper portion each recess 38, 40 is provided with an internal thread which is adapted to an external thread of a first clamp screw 42 and a second clamp screw 44, respectively. By screwing the clamp screws 42, 44 into the recesses 38 and 40, respectively, it is thus possible to secure the rods 14, 16 in the recesses 38, 40. As a matter of course, other types of clamp mechanisms may be used instead.

In the following various degrees of freedom will be explained which the connector 18 provides for. These degrees of freedom are advantageous because it is desirable that the surgeon is able to connect the rods 14, 16 to the connector 18 without a need to readjust the pedicle screws 12 or even the entire vertebrae. In this embodiment these degrees of freedom are only available during the surgery. Once the pedicle screws 12 are connected to the rods 14, 16 and the vertebrae to be treated are in the desired position, all moveable elements of the connector 18 will be fixed by the surgeon. A similar construction, but for a connector supporting only one seat member, is described in European patent application EP 09005904.9 filed Apr. 29, 2009.

In the embodiment shown the connector 18 is capable of being fixed in six different rotational positions with regard to the longitudinal axis 20 of the pedicle screw 12. This ability to rotate around the longitudinal axis 20 provides a first degree of rotational freedom. While the cylindrical portion 30 of the connector 18 remains centered with respect to the longitudinal axis 20 during such rotations, the projection 32 supporting the second seat member 36 swivels around the longitudinal axis 20, as is indicated in FIG. 1 by a cylinder 46.

Furthermore, the first seat member 34 is capable of being fixed in different rotational positions with regard to a rotational axis 48 which is not fixed, but can be polyaxially adjusted within a cone 50 of tilting angles. Thus the first seat member 34 can not only be rotated, but also tilted into various directions. In this embodiment the cone 50 has an axis of symmetry which coincides with the longitudinal axis 20 of the pedicle screw 12. In other embodiments this axis of symmetry runs parallel to the longitudinal axis 20 of the pedicle screw 12, or may even form an angle with this longitudinal axis 20.

The same degrees of freedom are available for the second seat member 36 so that also the second seat member 36 can be polyaxially adjusted within a cone 52 of tilting angles.

FIG. 2 is a sectional view through the connector 18 and an upper portion of the pedicle screw 12. The screw head 26 of the pedicle screw 12 is formed as an extension of the cylindrical portion 22 and comprises a circumferential groove 54 and a tapered end portion 56 which surrounds a hexagon socket 58.

The cylindrical portion 30 of the connector 18 is provided with a blind hole 60 whose diameter is selected such that the blind hole 60 can receive the screw head 26 with snug fit. At the ground of the blind hole 60 a hexagonal projection 62 is formed which exactly matches the shape of the hexagon socket 58 of the screw head 26.

In the wall defining the blind hole 60 a locking mechanism is accommodated which is configured to prevent movements of the pedicle screw 12 along its longitudinal axis 20 within the blind hole 60. In the embodiment shown the locking mechanism comprises two pins 64 loaded by springs 66, all of which are received in bores 68 provided in a circumferential groove 70 of the cylindrical portion 30. The springs 66 rest on plugs 72 which are pressed into the bores 68.

After the pedicle screw 12 has been screwed into a pedicle of a vertebra to be treated using a hexagon socket screw key adapted to the hexagon socket 58, the connector 18 is placed on the screw head 26. While the screw head 26 enters the blind hole 60, the tapered end portion 56 of the screw head 26 displaces the pins 64 that have been protruding into the blind hole 60. The surgeon may then select one of the six different rotational positions of the connector with regard to the screw head 26 by placing the projection 62 at the desired rotational position into the hexagon socket 58 of the screw head 26. If the projection 62 rests on the ground of the hexagon socket 58, the groove 54 of the screw head 26 will be at the height of the pins 64 which will then be pushed by the springs 66 into the groove 54 so as to achieve the desired locking effect. The projection 62 and the hexagon socket 58 then provide for a locking with regard to rotational movements, whereas the locking mechanism comprising the pins 64 and the groove 54 ensures that the connector 18 cannot move along the longitudinal axis 20 of the pedicle screw 12.

Removal of the connector is only possible if a suitable tool engages into the groove 70 provided in the cylindrical portion 30 of the connector 18. By pulling the tool, the pulling force exerted on the head member 28 of the connector 18 will eventually cause the portions of the pins 64 extending into the groove 54 to be sheared off so that the head member 28 can be released from the pedicle screw 12.

As a matter of course, other suitable locking mechanisms may be used instead. Furthermore, more sophisticated constructions that are capable of locking the screw head 26 in arbitrary rotational positions may be envisaged.

In the following details of the first and second seat members 34, 36 will be explained with reference to FIG. 2. Since both seat members 34, 36 have identical designs, the following description will only refer to the first seat member 34 for the sake of simplicity.

The first seat member 34 comprises a stepped bore, with an upper bore portion 74 having a larger diameter and a lower bore portion 76 having a smaller diameter. An upper half of the upper bore portion 74 is provided with an internal thread 77 which is adapted to an external thread 79 of the first clamp screw 42. The clamp screw 42 is provided at its upper end with indentations 80 adapted to receive a tip of a suitable screw driver.

A ground 82 of the lower bore portion 76 is concavely curved, with a center of curvature being arranged on the axis of symmetry 84 of the first seat member 34 which coincides, in the non-tilted position shown, with the longitudinal axis 20 of the pedicle screw 12. The lower portion of the first seat member 34 has a convex outer surface 86 and is received in a complementary concave recess 88 formed in the cylindrical portion 30 of the head member 28. The convex surface 86 and the concave recess 88 have centers of curvature which coincide with the center of curvature of the ground 82 of the lower bore portion 76.

The cylindrical bore portion 30 of the head member 28 further comprises a threaded bore 90 in which a first fixing screw 92 is screwed. A head 94 of the first fixing screw 92 rests on a curved washer 96 whose center of curvature also coincides with the center of curvature of the ground 82. The washer 96 has a central aperture 98 through which the bolt of the first fixing screw 92 extends.

The ground of the first seat member 34 is provided with a ground opening 100 which has, in the embodiment shown, the shape of a cone section. The outer diameter of the washer 96 is determined such that it sufficiently extends over the upper diameter of the ground opening 100, but is still significantly smaller then the diameter of the lower bore portion 76.

If the first fixing screw 92 is not tightened, the first seat member 34 is allowed to rotate around its axis of symmetry 84. Furthermore, the first seat member 34 as a hole, and thus also the rotational axis 48 coinciding with the axis of symmetry 84, can be tilted.

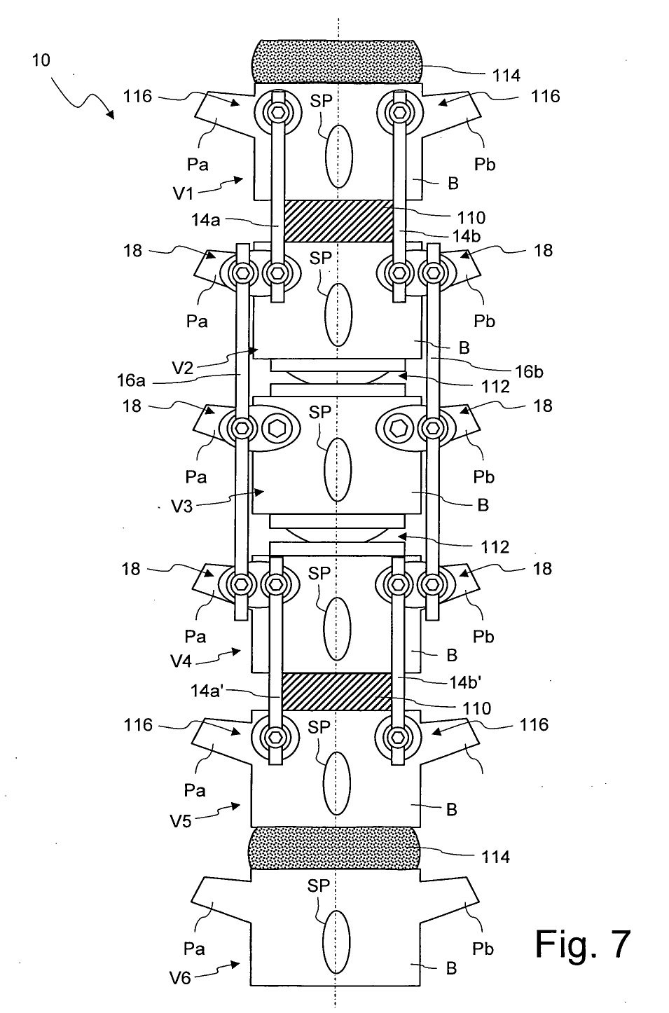

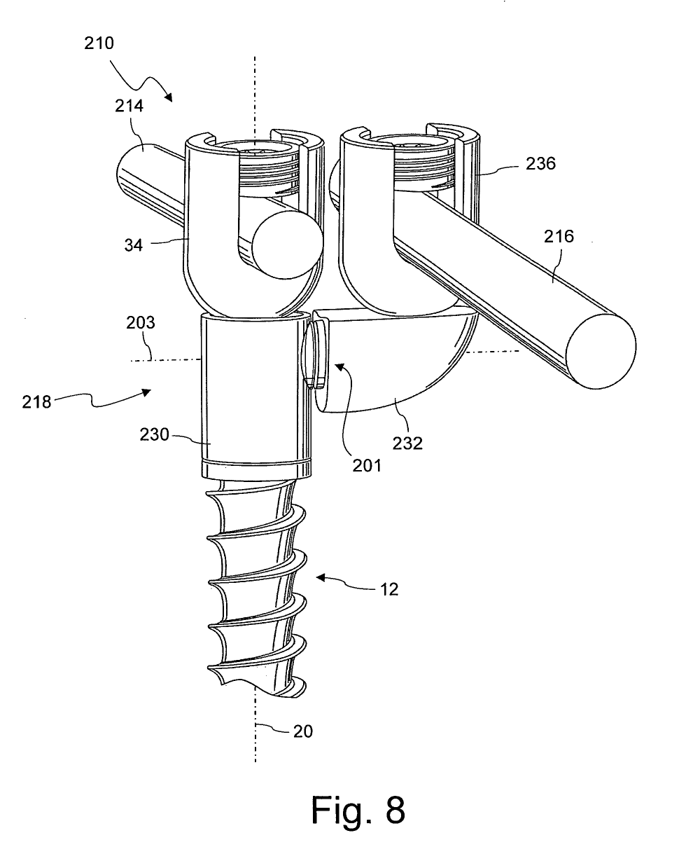

The tilted position can be seen in the cross-section of FIG. 8. Although FIG. 8 relates to a different embodiment, the first seat member 34 of the embodiment shown in FIG. 8 is identical to the first seat member 34 shown in FIG. 2. In FIG. 8 it can be seen that the washer 96 has slid along the ground 82 of the lower bore portion 76. The maximum tilt angle, i.e. the opening angle of the cone 50, is determined by the ratio of the diameters of the ground 82 and the washer 96. Also in the tilted position the first seat member 34 can still rotate around its axis of symmetry 84 when the first fixing screw 72 has not yet been tightened.

This design therefore enables a polyaxial adjustment of the first seat member 34 with respect to the cylindrical portion 30 of the head member 28. After the first seat member 34 is brought approximately in a rotational and tilting position that is required to receive the first rod 14, the latter may be inserted from above in the recess 38. This will often result in additional small movements of the first seat member 34. Then the rod 14 is carefully removed and the first fixing screw 92 is tightened. After tightening the first fixing screw 92, the first seat member 34 is fixed with respect to the head member 28 of the connector 18 and thus cannot perform any movement. Then the rod 14 is inserted again and secured with the help of the clamp screw 42.

As has been mentioned above, the second seat member 36 has the same design and function as the first seat member 34.

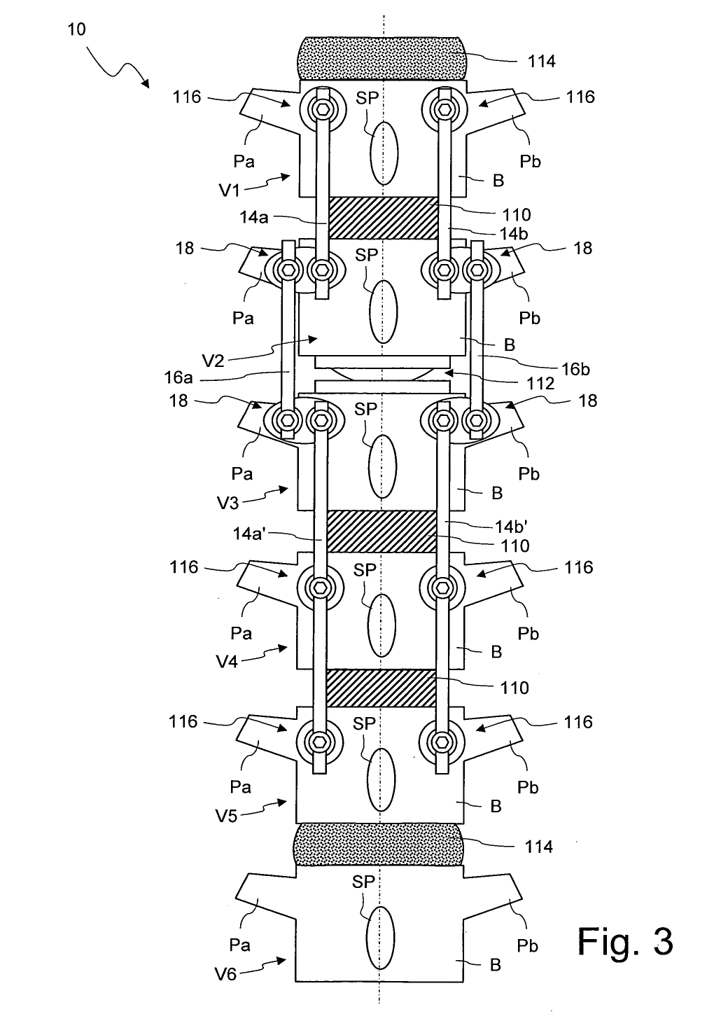

FIG. 3 is a simplified top view on a segment of the human spine in which the spine fixation system according to the present invention has been implanted. The spine segment comprises six vertebrae V1 to V6 each comprising a vertebral body B, a spinous process SP and two pedicles Pa, Pb.

The two vertebrae V1 and V2 shown on top of FIG. 3 are fused with the help of a fusion implant 110 which does not enable relative movement between the vertebrae V1 and V2. Between the vertebrae V2 and V3 a non-fusion implant 112 is inserted which enables the two vertebrae V2, V3 to perform relative movements. The non-fusion implant 112 may comprise a ball bearing, for example, and may be configured as described in WO 2007/003438 A2.

The three vertebrae V3, V4 and V5 are rigidly connected with two fusion implants 110. The vertebrae V5 and V6 are separated by the natural intervertebral disk 114.

The spine fixation system according to the present invention enables such a succession of fusion (rigid) and non-fusion (moveable) implants between adjacent vertebrae by using four connectors 18 which are fastened to the pedicles Pa, Pb of the vertebrae V2 and V3 with the help of the pedicle screw 12. The two rods 16a, 146 which connect the connectors 18 fastened to the vertebrae V2 and V3 are made of a resilient material such that the rods 16a, 16b bend if the relative position between the vertebra V1 and V2 is changed. This is shown in FIG. 4 which illustrates the configuration of the spine segment in a state of lateral flexion. Although the connectors 18 remain fixed, the resilient rods 14a, 14b enable a rotation of the vertebra V2 relative to the vertebra V3 around an axis which is perpendicular to the plane of the drawing sheet.

All other connectors 116 used for this spine segment have only one seat member so that only one rod can be connected to the respective pedicle screw. In this embodiment it is assumed that the connectors 116 are constructed similar to the connector 18, but without the projection 32 and without the second seat member 36. The seat members of the connectors 116 can then be polyaxially adjusted in the manner described above with references to FIGS. 1 and 2. Alternatively, connectors may be used for this purpose that are similar to the connectors 18 shown in FIGS. 1 and 2, but having only a seat member 36 positioned on the projection 32. Such connectors are described in the aforementioned European patent application EP 09005904.9 and offer a particular wide range of degrees of freedom.

The rods 14a, 14b and 14a′, 14b′ connected (also) to the connectors 116 having only one seat member are used to connect those pairs of vertebrae V1 and V2, V3 and V4, V4 and V5 which are separated by a fusion implant 110. These rods 14a, 14b and 14a′, 14b′ are therefore made of a rigid material having a significantly smaller bending stiffness than the rods 16a, 16b. Therefore the spine fixation system 10 rigidly fixes those vertebrae that are fused, but enables movement between vertebrae which are separated by a non-fusion implant 112.

It is to be understood that the rods 16a, 16b do not necessarily must have resilient properties which will result in a restoring moment exerted on the vertebrae V2 and V3. Instead rods 16a, 16b may be used that are flexible, but have no significant resilience. The flexibility is usually defined by the bending stiffness which is defined as the product of the area moment of inertia of the rods cross-section and its elastic modulus.

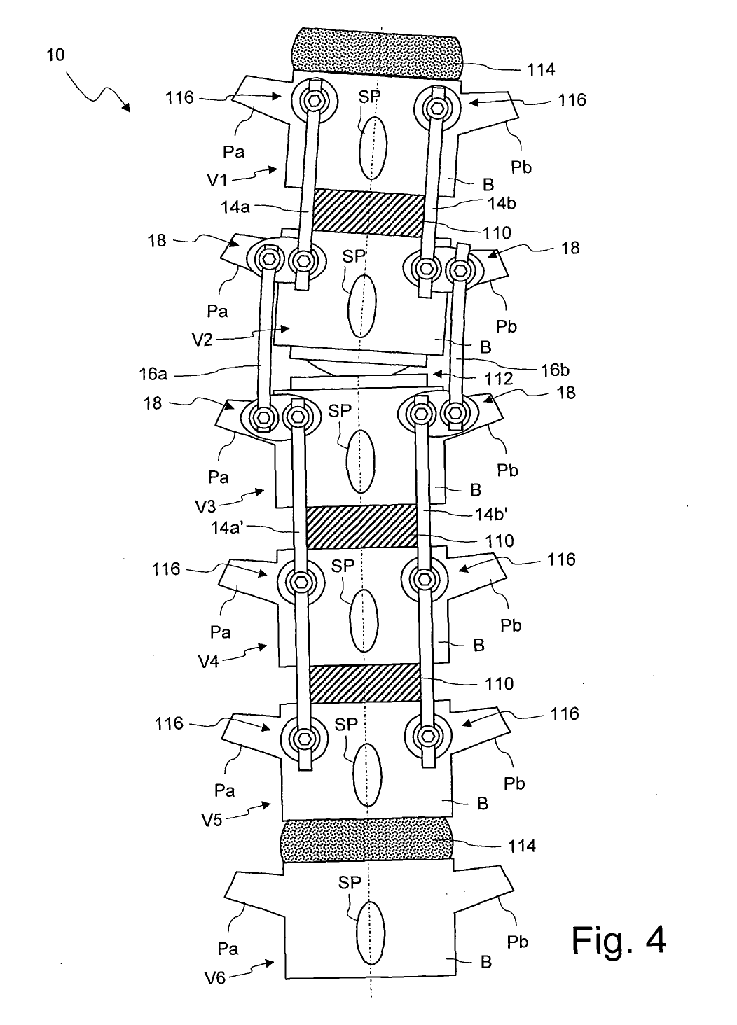

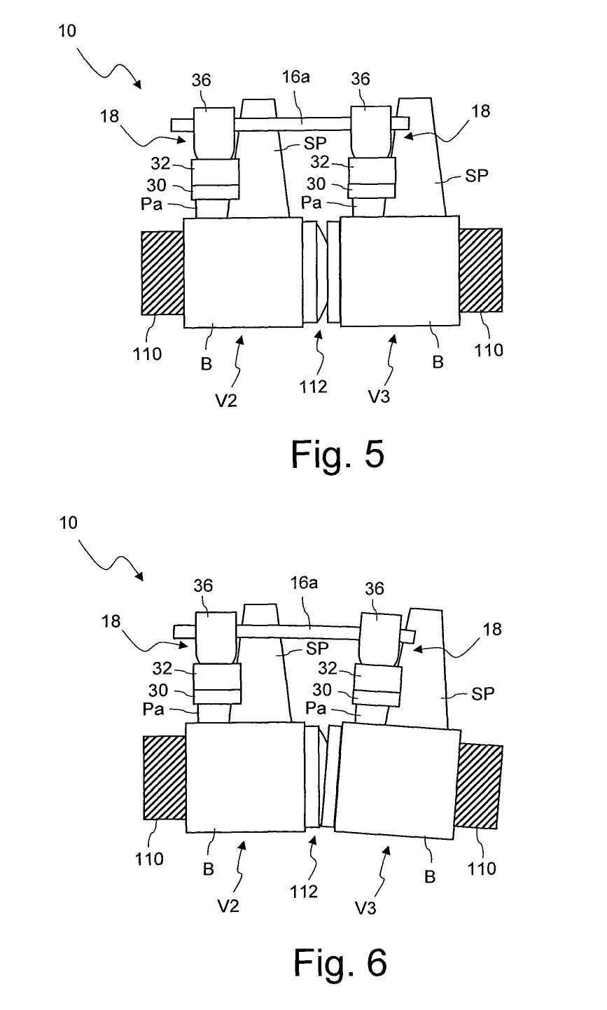

As a matter of course, the use of flexible rods 16a, 16b enables not only a lateral flexion as shown in FIG. 4, but almost any arbitrary relative movement between the vertebrae V2 and V3. This is illustrated in FIGS. 5 and 6 which are side views of the vertebrae V2 and V3 in a normal state and in an inclined state, respectively. For the sake of simplicity the rigid rods 14a, 14b and 14a′, 14b′ extending to the adjacent vertebrae V1 and V3, respectively, are not shown in FIGS. 5 and 6.

As can be seen from FIGS. 3 to 6, the flexible rods 16a, 16b are not only bent if the relative position of the vertebrae V2, V3 is changed, but are also subject to lengthwise compression or extension. This is due to the fact that the center of curvature of the bore bearing contained in the non-fusion implant 112 will usually not be located on one of the rods 16a, 16b. However, the resilient rods 16a, 16b will also have the ability to be compressed or expanded along their longitudinal axis to some extent.

FIG. 7 is a schematic top view of a segment of a human spine similar to the representation shown in FIG. 3. In this spine segment not only two, but three consecutive vertebrae V2, V3 and V4 are allowed to perform relative movements. To this end non-fusion implants 112 are arranged between adjacent pairs of vertebrae V2, V3 and V3, V4. Consequently, in this embodiment longer flexible rods 16a, 16b are used that extend not only over two, but over three consecutive vertebrae V2, V3 and V4.

### 2. Second Embodiment

FIG. 8 is a perspective view of a spine fixation system 210 according to second embodiment. Identical components are denoted with the same reference numerals as used before, whereas components which have only corresponding parts in the first embodiment described above are denoted by reference numerals augmented by 200.

One difference of the spine fixation system 210 to the spine fixation system 10 described above is that the projection 232 of the connector 218 is not integrally formed with the cylindrical portion 230. Instead, the projection is connected to the cylindrical portion 230 via a joint 201. The joint 201 enables the projection 232 to be rotated around a rotational axis 203 which runs perpendicular to the longitudinal axis 20 of the pedicle screw 12.

Another difference is that in the spine fixation system 210 according to the second embodiment both rods 214, 216 are both rigid rods, i.e. the rods 214, 216 have the same (high) bending stiffness. The degree of flexibility, which is achieved with the resilient rod 16 used in the spine fixation system 10 according to the first embodiment, is enabled according to the second embodiment by a flexible connection of the rigid rod 216 to the connector 218. More specifically, the second seat member 236 is attached to the projection 232 of the connector 218 such that the second seat member 236 is allowed to change its position with regard to the projection 232 even after the spine fixation system 210 has been implanted.

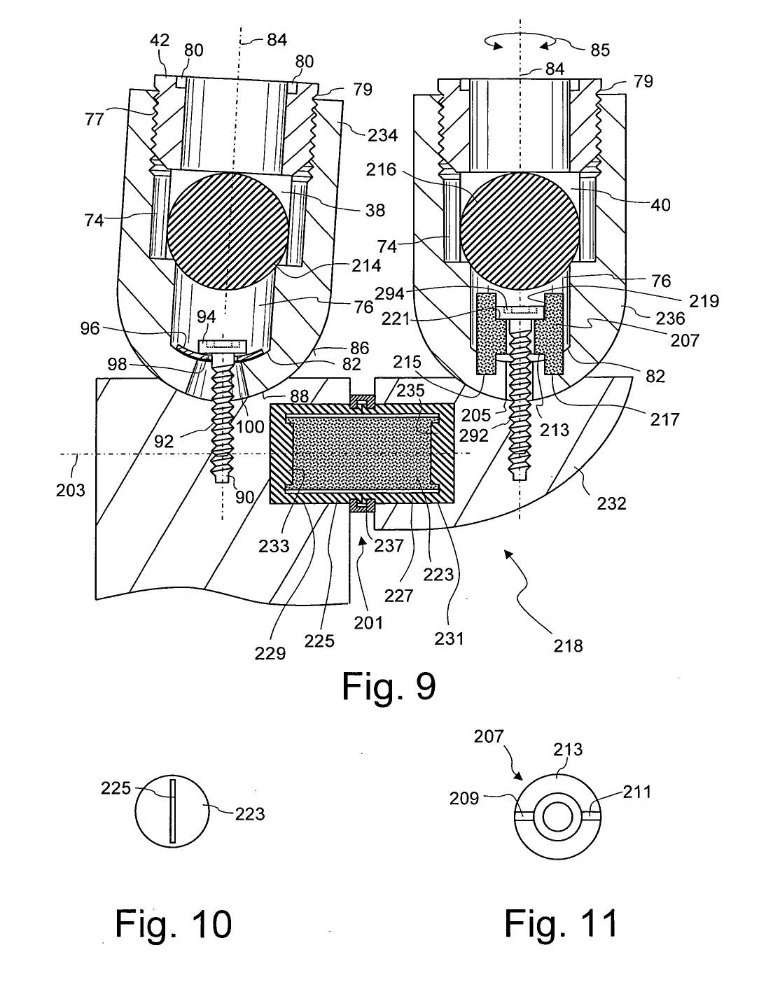

This will be explained in more detail with reference to FIG. 9 which is sectional view through the connector 218 and an upper portion of the pedicle screw 12.

The first seat member 234 is configured in the same way as the first seat member 34 of the spine fixation system 10 described above. However, the second seat member 236 is not in the same manner polyaxially adjustable as the first seat member 234. The opening 100 having the shape of a cone section is replaced by a cylindrical opening 205, and the washer 98 is replaced by a resilient ring 207. As can be seen in the bottom view shown in FIG. 11, the resilient ring 207 is provided at one end with two webs 209, 211 which axially project from a bottom surface 213 of the resilient ring 207. The webs 209, 211 engage into recesses 215, 217 which have a complementary shape with regard to the webs 209, 211 and are provided at the ground 82 of the lower bore portion 76.

At its opposite end the resilient ring 207 has a portion 219 in which the central bore has a greater diameter which is equal to the diameter of the head 294 of the fixing screw 292. In the assembled state the head 294 of the fixing screw 292 rests on a circumferential step 221 formed by the portion 219 and thus secures the second seat member 236 to the projection 232.

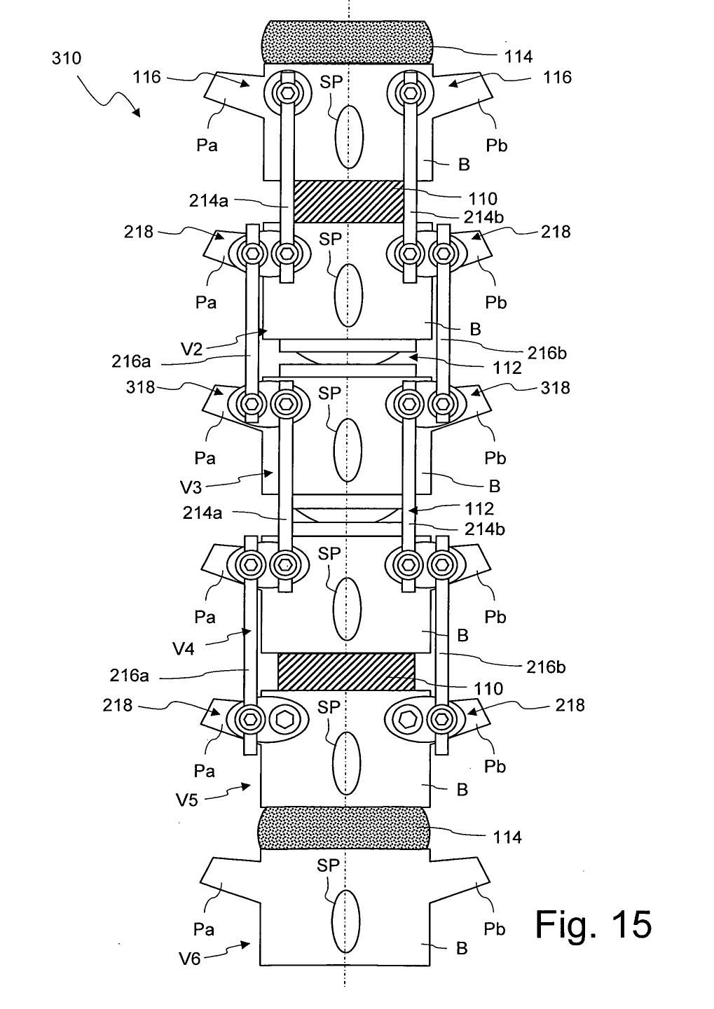

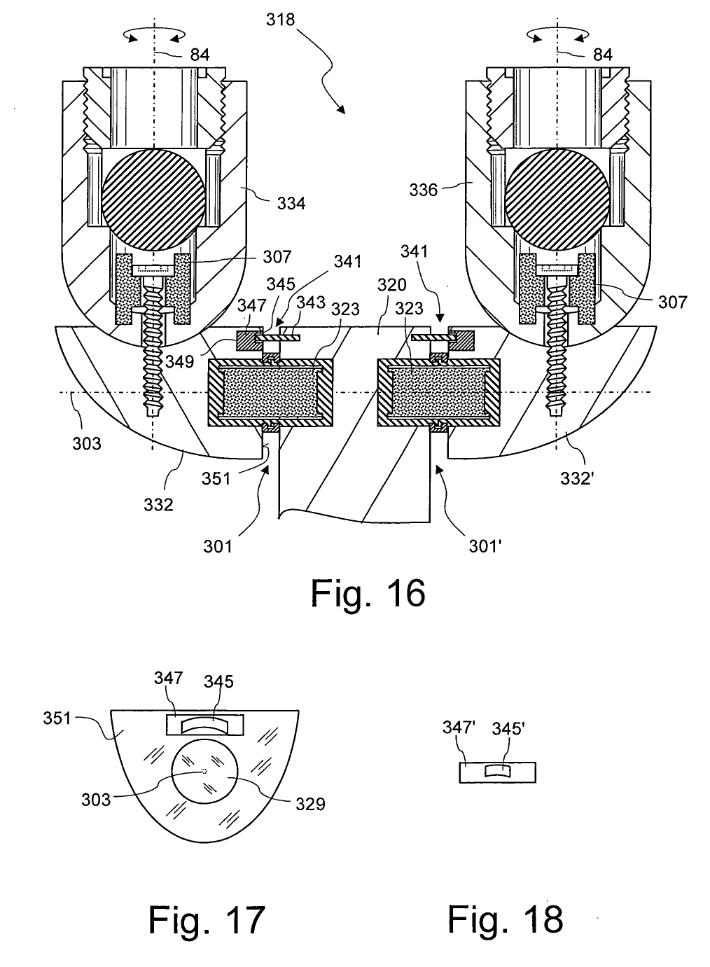

If the second seat member 236 is rotated around its axis of symmetry 84, the resilient ring 207 will undergo a torsion since its upper portion is frictionally engaged with the head 294 of the fixing screw 292, and its bottom portion is, via the webs 209, 211, rotationally fixed in the recesses 215, 217 provided in the second seat member 36. The restoring torque exerted by the resilient ring 207 increases with increasing rotational angles of the second seat member 236. By suitably selecting the material of the resilient ring 207, it is possible, at least to some extent, to delimit the range of rotations which the second seat member 236 is allowed to perform in relation to the projection 232 to a certain range, for example to 2° or 5°. More accurate angle range delimiters whose effect does not depend on the torque produced by the spine will be described further below with reference to FIGS. 16 to 18.

The second seat member 236 can also be tilted by small tilting angles around axes perpendicular to a longitudinal direction defined by the fixing screw 292. Thus the resilient ring 207 forms a joint that allows the second seat member 236 to perform rotational movements about three orthogonal axes with regard to the pedicle screw 292.

Depending on the material of the resilient ring 207, it may also be possible, by tightening the fixing screw 92 to different extents, to compress the resilient ring 207 so that its stiffness against torsion is changed. Thus the restoring moment exerted by the resilient ring 207 can be adjusted, at least to some extent, with the help of the fixing screw 92. This effect may be also used to change the range of allowed rotational angles.

The resilient ring 207 is received within the second seat member 236 such that it can easily be exchanged by a surgeon. If a plurality of resilient rings 207 is provided having a different stiffness against torsion, the surgeon may select a suitable ring 207 which provides, for the spine segment to be treated, the optimum restoring moments against rotations and tilting movements. The resilient ring 207 thus forms a first adjustable restoring force member which is configured to exert a restoring force acting against forces exerted by the spine and causing a position change of the second seat member 236 with respect to the pedicle screw 12 (or portions of the connector 218 rigidly connected to the pedicle screw 12).

The joint 201 connecting the cylindrical portion 230 to the projection 232 comprises a resilient member 223 which has a generally cylindrical shape. As can be seen in the top and bottom view of FIG. 10, the resilient member 223 is provided at its top and bottom surface with a slit-like groove 225. The resilient member 223 is received in two cupular holders 225, 227 having identical shapes. Each cupular holder 225, 227 is squeezed in cylindrical recesses 229, 231 provided in the cylindrical portion 230 and the projection 232, respectively. The cupular holders 225 are provided at their bottom with a ridge 233, 235 having a complementary shape with regard to the groove 225 at the opposite ends of the resilient members 223. At their upper end the cupular holders 225, 227 are provided on their outer surfaces with circumferential grooves into which a spring ring 237 engages.

With the ridges 233, 235 engaging into the grooves 225, 227 of the resilient member 223, the latter is rotationally fixed within the cupular holders 225, 227. Since the cupular holders 225, 227 are also fixedly received in the recesses 229, 231, the projection 232 can perform rotational movements about the rotational axis 203 because the cupular holders 225, 227 are allowed to rotate with regard to the spring ring 237. The torsion of the resilient member 223, which takes place if the projection 232 rotates with regard to the cylindrical portion 230, results in an increasing restoring torque the greater the angle of rotation is.

The neutral position of the projection 232 with regard to the cylindrical portion 230 is defined by the state in which the resilient member 223 is not subjected to torsion. This state is, in turn, defined by the position of the grooves 225 provided at the opposite ends of the resilient member 223. Thus different neutral positions may be defined by selecting a suitable resilient member 223 out of a set of different resilient members having different relative angular arrangements of the grooves 225 provided at their opposite ends. The set of resilient members 223 may also differ with regard to the resilience of the resilient member 223, i.e. the stiffness against torsion which defines the restoring torque provided by the joint 201. The resilient ring member 223 thus forms a second adjustable restoring force member which is configured to exert a restoring force acting against forces exerted by the spine and causing a position change of the second seat member 236.

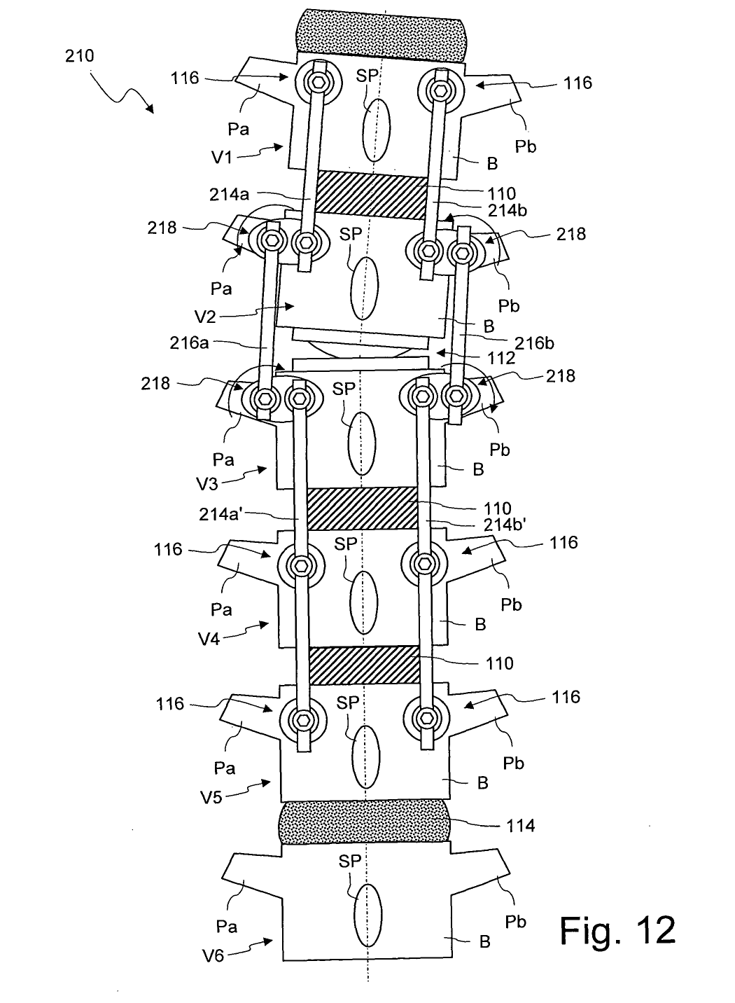

FIG. 12 is a schematic top view of a segment of a human spine in which the spine fixation system 210 has been implanted, with two adjacent vertebrae being in a state of lateral flexion similar to what is shown in FIG. 4. In this embodiment the lateral flexure is not enabled by flexible rods. Instead, since the second seat members 236 of the connectors 218 are allowed to perform rotations around the axis 84 as indicated by a double arrow 85 in FIG. 9, the rigid rods 216a, 216b rotate with regard to the connectors 218 if the vertebrae V2, V3 perform a relative rotational movement enabled by the non-fusion implant 112.

The restoring force which the spine fixation system 210 exerts against such lateral flexure will mainly depend on the properties of the resilient ring 207 which undergoes torsion in the case of lateral flexure of the vertebrae V2, V3.

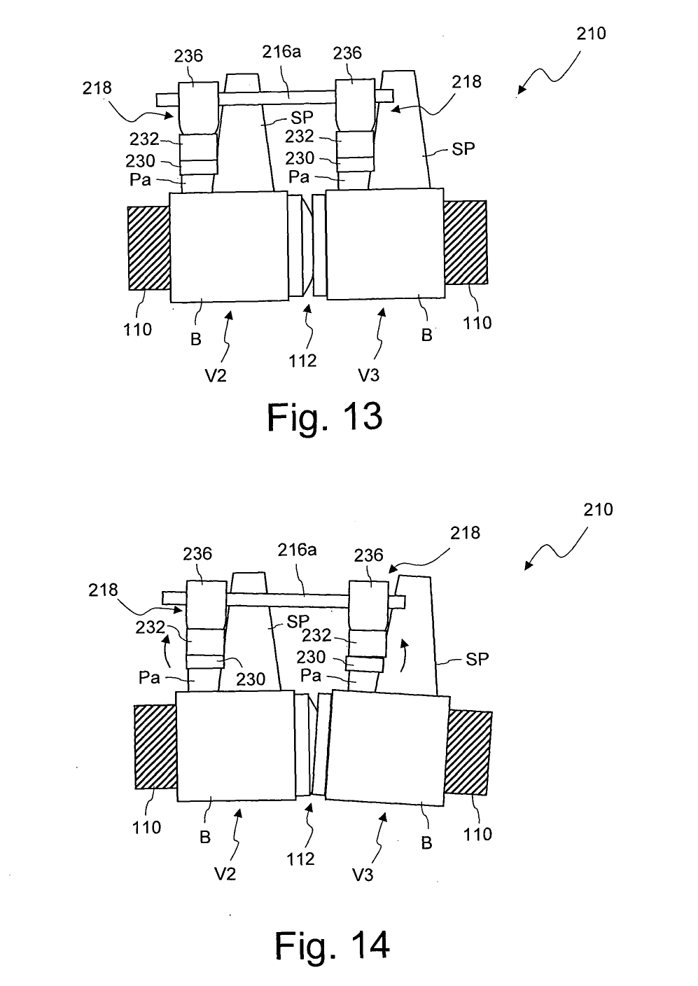

FIGS. 13 and 14 illustrate, in drawings similar to FIGS. 5 and 6, how the spine fixation system 210 also enables a controlled inclination or reclination of the vertebrae V2, V3. As can be seen in FIG. 14, the projections 232 which support the second seat member 236s of the connectors 218, rotate relative to the cylindrical portions 230, as is indicated by arrows in FIG. 14. The restoring torque against such rotations of the joints 201 is mainly determined by the properties of the resilient members 223.

A relative rotation of the vertebrae V2, V3 as shown in FIGS. 12 and 14 will usually require that the rods 216a, 216b are allowed to perform small longitudinal movements within at least one second seat member 236. Such movements may be enables, for example, by not fully tightening the clamp screws 44. However, more sophisticated measures may be envisaged as well in this respect.

In the second embodiment described the second seat member 236 is allowed to perform two different rotational movements after it has been implanted. It may also be envisaged to enable rotational movements around three (orthogonal) axes. Furthermore, if no restoring torques are desired for these rotational movements, connectors may be used that do not contain any resilient elements, but only simple joints comprising shafts, ball bearing etc.

### 3. Third Embodiment

FIG. 15 is a schematic top view of a segment of a human spine in which a spine fixation system 310 has been implanted. Similar to the spine segment shown in FIG. 7, not only two, but three consecutive vertebrae V2, V3 and V4 are allowed to perform relative movements by the use of two non-fusion implants 112. However, the spine fixation system does not enable these relative movements with the help of flexible rods, but with the flexible connectors 218 described above with reference to FIGS. 8 to 14 and an additional type of connector 318.

This additional type of connector 318 is fastened, via pedicle screws 12, to the vertebra V3 and allows to change the position not of only one, but of both seat members 334, 336 after the spine fixation system 310 has been implanted. This is necessary because not only the rigid rods 216a, 216b, but also the rigid rods 214a′, 214b′, secured to the connectors 318 must be able to perform articulating movements with respect to the vertebra V3. Such a connector 318 may be realized simply by adopting the mechanism used for the second seat member 236 in the second embodiment also for the first seat member 234, and by arranging both seat members on projections that can be tilted around the axis 303.

An exemplary configuration for such a connector 318 is shown in the sectional view of FIG. 15. Both seat members 334, 336 can be rotated around their axes of symmetry 84 against the resistance exerted by the resilient rings 307. A second degree of rotational freedom is provided by the two joints 301, 301′ which connect projections 332, 332′ to a cylindrical portion 320 which is fastened to the pedicle screw 312.

The connector 18 further comprises, for each seat member 334, 336 an adjustable angular range delimiter 341 which is configured to delimit a range of allowed rotational angles of the seat members 334 and 336, respectively, with respect to the rotational axis 303. Each angular range delimiter 341 comprises a cam 343 formed by a rigid pin which is fixed in the cylindrical portion 320 of the connector 318. The cam 343 reaches into a groove 345 provided in an insert 347 which is fixedly received in a complementary recess 349 provided at a planar face 351 of the projections 332 and 332′. As can be seen in the side view on the planar face 351 shown in FIG. 17, the groove 345 in the insert 347 has the shape of a ring segment, wherein the center of curvature of the ring segment coincides with the rotational axis 303. The range of allowed rotational angles, by which the seat members 334, 336 can rotate around the axis 303, is defined by the length of the groove 345.

For adjusting the range of allowed rotational angles, the insert 347 may be replaced by another insert having a groove with a different length. A suitable insert 347′ having a shorter groove 345′ is shown, in a side view similar to FIG. 17, in FIG. 18. To this end a mechanism (not shown) for snap-on mounting may be provided that enables an easy mounting of the insert 347′ in the recess 349 in the projections 332, 332′. This type of range adjustment therefore involves the selection of one out of a set of different inserts 347.

It is also envisaged to use more sophisticated mechanisms for adjustably delimiting the range of allowed rotational angles. In particular, adjusting crews of similar elements may be used to this end so that also a continuous adjustment is possible.

Adjustable angular range delimiters may also be used to delimit the range of allowed rotational angles of the seat members 334, 336 with regard to the axis 84. If a third rotational axis or another degree of freedom is provided, an adjustable angular range delimiter may be envisaged as well. As a matter of course, also the connector 218 of the second embodiment may comprise an adjustable angular range delimiters.

The above description of the preferred embodiments has been given by way of example. From the disclosure given, those skilled in the art will not only understand the present invention and its attendant advantages, but will also find apparent various changes and modifications to the structures and methods disclosed. The applicant seeks, therefore, to cover all such changes and modifications as fall within the spirit and scope of the invention, as defined by the appended claims, and equivalents thereof.

1. A spine fixation system, comprising:
a) two rods which are configured to extend over a portion of the spine,
b) a plurality of fasteners, wherein each fastener has a longitudinal axis and is configured to be secured to a vertebra to be treated,
c) a connector which
is attached to, or is capable of being attached to, one of the fasteners and
comprises two seat members each being configured to be connected to one of the two rods, wherein
the position of at least one of the two seat members relative to the fastener, to which the connector is attached, is, after the spine fixation system has been completely implanted in the human body, allowed to change in response to forces exerted by the portion of the spine.

2. The system of claim 1, wherein the connector comprises a joint that allows changes of the position of the at least one seat member after the spine fixation system has been completely implanted in the human body.

3. The system of claim 1, wherein the at least one seat member is allowed to perform rotational movements about at least two different axes with regard to the fastener to which the connector is attached.

4. The system of claim 3, wherein the at least one seat member is allowed to perform rotational movements about a first axis and a second axis, and wherein the connector comprises a body portion, through which the longitudinal axis of the fastener, to which the connector is connected, extends, and an arm member which projects from the body portion and supports the at least one seat member, wherein the first axis extends through the at least one seat member and the arm member and the second axis extends through the arm member and the body portion.

5. The system of claim 1, comprising a range delimiter which is configured to delimit a range of allowed position changes.

6. The system of any claim 1, comprising a restoring force member which is configured to exert a restoring force acting against the forces exerted by the portion of the spine and causing a position change of the at least one seat member.

7. (canceled)

8. The system of claim 1, wherein at least one of the two seat members is capable of being fixed in different rotational positions with regard to a rotational axis that is, for enabling the at least one seat member to be polyaxially adjusted, capable of being fixed in different tilting positions at least within a cone of tilting angles.

9. The system of claim 1, comprising a further connector which is attached to, or is capable of being attached to, one of the fasteners and which comprises exactly one seat member being configured to be connected to one of the two rods, wherein the position of the seat member is, after the spine fixation system has been completely implanted in the human body, fixed with regard to the fastener to which the further connector is attached.

10. (canceled)

11. A spine fixation system, comprising:
a) a first rod that connects a first vertebra to a second vertebra but not to a third vertebra,
b) a second rod that connects the second vertebra to the third vertebra but not to the first vertebra,
wherein the spine fixation system is configured such that the first vertebra, but not the third vertebra, is allowed to move relative to the second vertebra after the spine fixation system has been completely implanted in the human body.

12. The system of claim 11, wherein the first rod has a smaller bending stiffness than the second rod.

13. The system of claim 11, comprising fasteners, which are secured to the vertebrae, wherein the first rod is flexibly connected to fasteners that are secured to the first and second vertebrae.

14. The system of claim 13, comprising connectors, which are attached to the fasteners and comprise seat members that are connected to the rods, wherein the position of the seat members that are connected to the first rod are, after the spine fixation system has been completely implanted in a human body, allowed to change in response to forces exerted by the first and second vertebrae.

15. A spine fixation system, comprising:
a) two rods which are configured to extend over a portion of the spine,
b) a plurality of fasteners, wherein each fastener has a longitudinal axis and is configured to be secured to a vertebra to be treated,
c) a connector which
is attached to, or is capable of being attached to, one of the fasteners and
comprises two seat members each being configured to be connected to one of the two rods,
Wherein the position of at least one of the two seat members relative to the fastener, to which the connector is attached, is, after the spine fixation system has been completely implanted in the human body, allowed to change in response to forces exerted by the portion of the spine,
and wherein the connector comprises a restoring force member which is configured to exert a restoring force acting against the forces exerted by the portion of the spine and causing a position change of the at least one seat member.
